(19) United States
(12) Patent Application Publication
(10) Pub. No.: US 2006/0079537 A1
(43) Pub. Date: Apr. 13, 2006

(54) 2-Substitutued triazolopyrimidines, methods and intermediate products for the production thereof, the use of the same controlling pathogenic fungi, and agents containing said compounds

(76) Inventors: Jordi Tormo I Blasco, Laudenbach (DE); Carsten Blettner, Mannheim (DE); Bernd Muller, Frankenthal (DE); Markus Gewehr, Kastellaun (DE); Wassilios Grammenos, Ludwigshafen (DE); Thomas Grote, Wachenheim (DE); Andreas Gypser, Mannheim (DE); Joachim Rheinheimer, Ludwigshafen (DE); Peter Schafer, Ottersheim (DE); Frank Schieweck, Hebheim (DE); Anja Schwogler, Mannheim (DE); Eberhard Ammermann, Heppenheim (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(21) Appl. No.: 10/531,898

(22) PCT Filed: Nov. 14, 2003
(86) PCT No.: PCT/EP03/12774

§ 371 Date: Apr. 21, 2005

(30) Foreign Application Priority Data
Nov. 15, 2002 (DE) 102 53 592.2
Dec. 6, 2002 (DE) 102 57 394.8

(51) Int. Cl. A61K 31/519; C07D 487/04

(52) U.S. Cl. 514/259.31; 544/263

(57) ABSTRACT

2-Substituted triazolopyrimidines of the formula I, 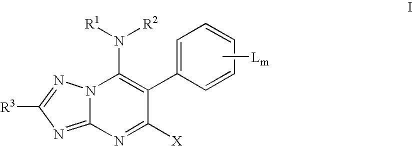 in which the substituents are as defined below:
L are halogen, cyano, nitro, alkyl, alkenyl, alkynyl, haloalkyl, haloalkenyl, alkoxy, alkenyloxy, alkynyloxy, haloalkoxy, —C(═O)-A or S(═O)ₚA′; A and A′ are as defined in the description; p is 0, 1 or 2; m is 0, 1, 2, 3, 4 or 5; X is cyano, alkyl, haloalkyl, alkoxy or haloalkoxy;
R¹,R² are hydrogen, alkyl, haloalkyl, cycloalkyl, halocycloalkyl, alkenyl, alkadienyl, haloalkenyl, cycloalkenyl, alkynyl, haloalkynyl or cycloalkynyl, phenyl, naphthyl or a five- to ten-membered saturated, partially unsaturated or aromatic heterocycle which contains one to four heteroatoms from the group consisting of O, N and S,
   R¹ and R² together with the nitrogen atom to which they are attached may also form a five- or six-membered ring which may be interrupted by an atom from the group consisting of O, N and S,
   where R¹ and/or R² may be substituted as defined in the description; and
R³ is cyano, hydroxyl, alkoxy, alkenyloxy, haloalkoxy, haloalkenyloxy, NR¹R² or S(O)ₙR³¹, where n and R³¹ are as defined in the description; Processes and intermediates for preparing these compounds, compositions comprising them and their use for controlling phytopathogenic harmful fungi are described.

The invention relates to 2-substituted triazolopyrimidines of the formula I,

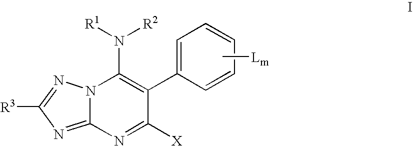

in which the substituents are as defined below:
L independently of one another are halogen, cyano, nitro, C₁-C₆-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, —C(═O)-A or S(═O)ₚA′;
   A is hydrogen, hydroxyl, C₁-C₈-alkyl, C₂-C₈-alkenyl, C₁-C₈-alkoxy, C₁-C₆-haloalkoxy, C₁-C₈-alkylamino or di-(C₁-C₈-alkyl)amino;
   A′ is hydrogen, C₁-C₈-alkyl or C₁-C₆-haloalkyl;
   p is 0, 1 or 2;
m is 0, 1, 2, 3, 4 or 5;
X is cyano, C₁-C₄-alkyl, C₁-C₄-haloalkyl, C₁-C₄-alkoxy or C₁-C₂-haloalkoxy;
R¹,R² independently of one another are hydrogen, C₁-C₈-alkyl, C₁-C₈-haloalkyl, C₃-C₆-cycloalkyl, C₃-C₆-halocycloalkyl, C₂-C₈-alkenyl, C₄-C₁₀-alkadienyl, C₂-C₈-haloalkenyl, C₃-C₆-cycloalkenyl, C₂-C₈-alkynyl, C₂-C₈-haloalkynyl or C₃-C₆-cycloalkynyl, phenyl, naphthyl, or a five- to ten-membered saturated, partially unsaturated or aromatic heterocycle which contains one to four heteroatoms from the group consisting of O, N and S,
   R¹ and R² together with the nitrogen atom to which they are attached may also form a five- or six-membered ring which may be interrupted by an atom from the group consisting of O, N and S and/or may carry one or more substituents from the group consisting of halogen, C₁-C₆-alkyl, C₁-C₆-haloalkyl and oxy-C₁-C₃-alkyleneoxy or in which a nitrogen atom and an adjacent carbon atom may be linked by a C₁-C₄-alkylene chain;
   where R¹ and/or R² may be substituted by one to four identical or different groups R^{a}:
   R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₃-C₆-cycloalkyl, phenyl, naphthyl, a five- to ten-membered saturated, partially unsaturated or aromatic heterocycle which contains one to four heteroatoms from the group consisting of O, N and S,
      where these aliphatic, alicyclic or aromatic groups for their part may be partially or fully halogenated or may carry one to three groups R^{b}:
      R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups in these radicals contain 2 to 8 carbon atoms;
      and/or one to three of the following radicals:
      cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups; and
R³ is cyano, hydroxyl, C₁-C₈-alkoxy, C₃-C₈-alkenyloxy, C₁-C₈-haloalkoxy, C₃-C₈-haloalkenyloxy, NR¹R² or S(O)ₙR³¹;
   n is 0, 1 or 2;
R³¹ is hydrogen, hydroxyl, C₁-C₈-alkyl, C₂-C₈-alkenyl or —C(═O)-A.

Moreover, the invention relates to processes and intermediates for preparing these compounds, to compositions comprising them and to their use for controlling phytopathogenic harmful fungi.

6-Phenyl-7-aminotriazolopyrimidines are known in a general manner from EP-A 71 792 and EP-A 550 113. Triazolopyrimidines substituted in the 2-position are disclosed in EP-A 71 792, EP-A 141 317, WO 02/88126 and WO 02/88127. The compounds described in the publications mentioned are suitable for controlling harmful fungi.

However, in many cases their action is unsatisfactory. It is an object of the present invention to provide compounds having improved activity and/or the broader activity spectrum.

We have found that this object is achieved by the compounds defined at the outset. Furthermore, we have found processes and intermediates for the preparation, compositions comprising them and methods for controlling phytopathogenic harmful fungi using the compounds I.

The compounds of the formula I differ from those in the publications mentioned above by the substituent in the 2-position or 5-position.

Compared to the known compounds, the compounds of the formula I have increased activity against phytopathogenic harmful fungi.

The compounds according to the invention can be obtained by different routes.

The present invention preferably provides compounds of the formula I.1 in which the variables and the index are as defined for formula I.

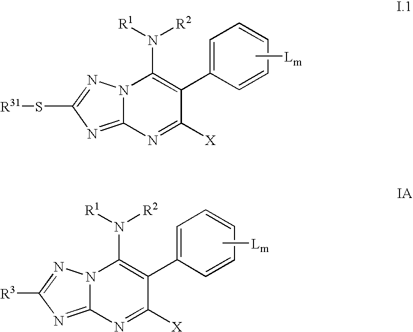

The invention furthermore preferably provides compounds of the formula IA in which R³ is cyano, hydroxyl, C₁-C₈-alkoxy, C₁-C₈-haloalkoxy, C₃-C₈-haloalkenyloxy or NR¹R² and Lₘ, R¹, R² and X are as defined in formula I.

Thio compounds of the formula I.1 in which X is cyano, alkoxy or haloalkoxy are advantageously obtained by reacting halogen compounds of the formula II in which Hal is preferably chlorine with compounds M-X′ (formula III).

Depending on the meaning of the group X′ to be introduced, compounds III are inorganic cyanides, alkoxides or haloalkoxides. The reaction is advantageously carried out in the presence of an inert solvent. The cation M in formula III is of minor importance; for practical reasons, preference is usually given to ammonium, tetraalkylammonium or alkali metal or alkaline earth metal salts.

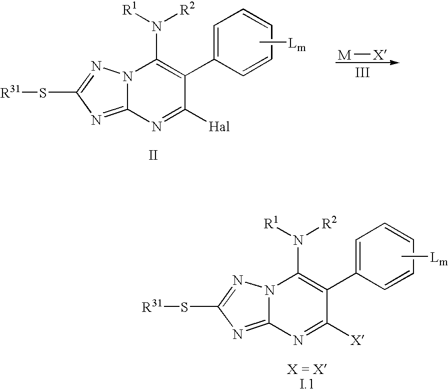

Here the reaction temperature is usually from 0 to 120° C., preferably from 10 to 40° C. [cf. J. Heterocycl. Chem. 12, (1975), 861-863].

Suitable solvents include ethers, such as dioxane, diethyl ether and, preferably, tetrahydrofuran, halogenated hydrocarbons, such as dichloromethane, and aromatic hydrocarbons, such as toluene.

Thioalkyl compounds of the formula II are known per se from WO 02/88127.

Compounds of the formula I.1 in which X is C₁-C₄-alkyl (formula I.1′) can be prepared from compounds II in which Hal is in particular chlorine and malonates of the formula IV. In formula IV, X″ is hydrogen or C₁-C₃-alkyl and R is C₁-C₄-alkyl. Compounds IV are converted into compounds of the formula V and decarboxylated to give compounds I.1′ [cf. U.S. Pat. No. 5,994,360].

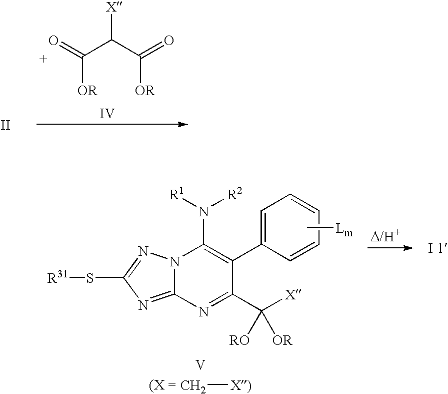

The malonates IV are known from the literature [J. Am. Chem. Soc., 64, (1942), 2714; J. Org. Chem., 39, (1974) 2172; Helv. Chim. Acta, 61, (1978), 1565], or they can be prepared in accordance with the literature cited.

The subsequent hydrolysis of the ester V is carried out under generally customary conditions; depending on the various structural elements, alkaline or acidic hydrolysis of the compounds V may be advantageous. Partial or complete decarboxylation to I.1′ may already occur under the conditions of ester hydrolysis.

Decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitrites, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preferences the reaction is carried out in hydrochloric acid or acetic acid. It is also possible to use mixtures of the solvents mentioned.

Compounds of the formula I.1 in which X is C₁-C₄-alkyl or C₁-C₄-haloalkyl can advantageously also be obtained by the synthesis route below:

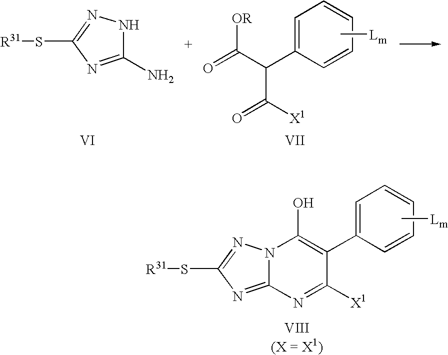

The 5-alkyl-7-hydroxy-6-phenyltriazolopyrimidines VIII are obtained from diketones VII. In formula VII, X¹ is C₁-C₄-alkyl or C₁-C₄-haloalkyl. Using the easily obtainable 2-phenylacetoacetic esters (VII where X¹═CH₃), the 5-methyl-7-hydroxy-6-phenyltriazolopyrimidines are obtained [cf. Chem. Pharm. Bull., 9, (1961) 801]. The preparation of the starting materials VII is advantageously carried out under the conditions described in EP-A 10 02 788.

The 5-alkyl-7-hydroxy-6-phenyltriazolopyrimidines obtained in this manner are reacted with halogenating agents to give the 7-halotriazolopyrimidines of the formula IX. Preference is given to using chlorinating or brominating agents such as phosphorus oxybromide, phosphorus oxychloride, thionyl chloride, thionyl bromide or sulfuryl chloride. The reaction can be carried out in the absence or in the presence of a solvent. Customary reaction temperatures are from 0 to 150° C. or preferably from 80 to 125° C. [cf. EP-A 770 615].

The 5-Alkyl-7-halo-6-phenyltriazolopyrimidines of the formula IX are reacted further with amines of the formula X in which R¹ and R² are as defined in formula I to give compounds of the formula I.1.

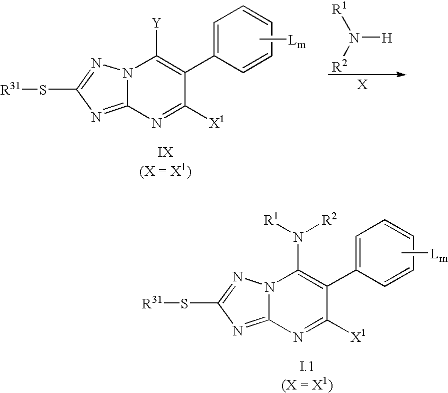

This reaction is advantageously carried out at from 0° C. to 70° C., preferably at from 10° C. to 35° C., preferably in the presence of an inert solvent, such as an ether, for example dioxane, diethyl ether or in particular tetrahydrofuran, a halogenated hydrocarbon, such as dichloromethane, or an aromatic hydrocarbon, such as, for example, toluene [cf. WO 98/46608].

It is preferred to use a base, such as a tertiary amine, for example triethylamine, or an inorganic amine, such as potassium carbonate; it is also possible for excess amine of the formula X to serve as base.

Compounds of the formula I.1 in which X is alkyl or haloalkyl can also be obtained by coupling 5-halotriazolopyrimidines of the formula II with organometallic reagents of the formula XI in which X is C₁-C₄-alkyl or C₁-C₄-haloalkyl. In one embodiment of this process, the reaction is carried out with transition metal catalysis, such as Ni or Pd catalysis.

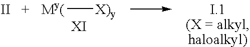

In formula XI, M is a metal ion of valency y, such as, for example, B, Zn or Sn. This reaction can be carried out, for example, according to the methods below: J. Chem. Soc. Perkin Trans. 1, (1994), 1187, ibid. 1, (1996), 2345; WO 99/41255; Aust. J. Chem., 43, (1990), 733; J. Org. Chem., 43, (1978), 358; J. Chem. Soc. Chem. Commun. (1979), 866; Tetrahedron Lett., 34, (1993), 8267; ibid., 33, (1992), 413.

Compounds of the formula I in which R³ is S(O)₁₋₂ R³¹ are obtained by oxidizing the corresponding thio compounds I.1.

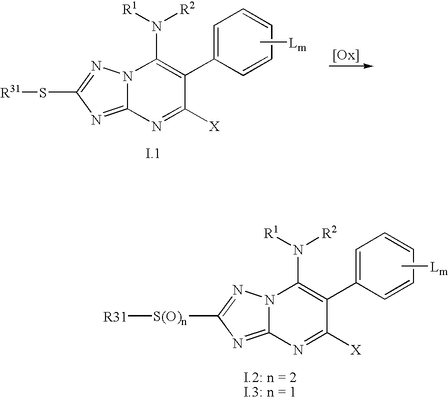

The oxidation of the thiolates I.1 to sulfones I.2 or sulfoxides I.3 is usually carried out at temperatures of from −40° C. to 60° C., preferably from −40° C. to 40° C., in an inert organic solvent [cf. WO 94/14761; Synth. Commun., 16, (1986), 233ff.]. Suitable oxidizing agents are, for example, inorganic peroxides, such as hydrogen peroxide, or peroxocarboxylic acids, such as peracetic acid, or perbenzoic acids, in particular meta-chloroperbenzoic acids. The starting materials are generally reacted with one another in equimolar amounts. In terms of yield, it may be advantageous to employ an excess of oxidizing agent, based on II.1.

Compounds of the formula I in which R³ is S—R³¹ (formula I.1) are also useful intermediates for preparing further compounds I.1.

For practical reasons, for preparing compounds IA, it is preferred to use intermediates in which R³¹ is methyl.

Compounds of the formula I in which R³ is not S(O)ₙR³¹ are advantageously obtained by reacting sulfones of the formula I.2 under basic conditions with compounds of the formula XII. Depending on the nature of group R³, compounds XII are cyanides, hydroxides, alkoxides or amines. The cation M in formula XII is of minor importance; for practical reasons, preference is usually given to ammonium, tetraalkylammonium or alkali metal or alkaline earth metal salts.

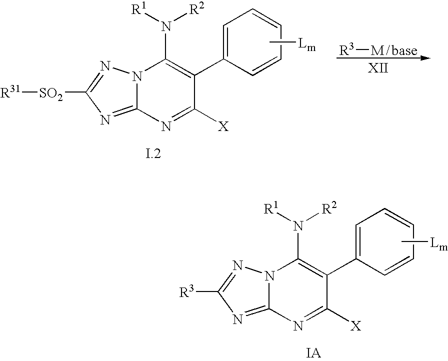

This reaction is duly carried out at temperatures of from −20° C. to 120° C., preferably from 0° C. to 25° C., in an inert organic solvent in the presence of a base [cf. Heteroat. Chem. (2000), 313].

Suitable solvents are aliphatic or aromatic hydrocarbons, such as benzene, toluene, o-, m- and p-xylene, halogenated hydrocarbons, ethers, such as diethyl ether, diisopropyl ether, tert-butyl methyl ether, dioxane, anisole and tetrahydrofuran, nitriles, ketones, alcohols, and also dimethyl sulfoxide, dimethylformamide and dimethylacetamide, with particular preference dimethyl sulfoxide, dioxane and benzene. It is also possible to use mixtures of the solvents mentioned.

Suitable bases are, in general, inorganic compounds, such as alkali metal and alkaline earth metal hydrides, such as lithium hydride, sodium hydride, potassium hydride and calcium hydride, moreover organic bases, for example tertiary amines, and also bicyclic amines. Particular preference is given to sodium hydrides. The bases are generally employed in catalytic amounts; however, they can also be employed in equimolar amounts, in excess or, if appropriate, as solvents.

The starting materials are generally reacted with one another in equimolar amounts. In terms of yield, it may be advantageous to employ an excess of XII, based on I.2.

A particularly advantageous access to the intermediates of the formula II in which R³¹ is methyl is given by the routes below:

Starting with 3-thiomethyl-5-aminotriazole of the formula VI′ and using appropriately substituted phenylmalonates of the formula XIII in which R is alkyl, preferably C₁-C₆-alkyl, in particular methyl or ethyl, the dihydroxytriazolopyrimidines XIV are prepared.

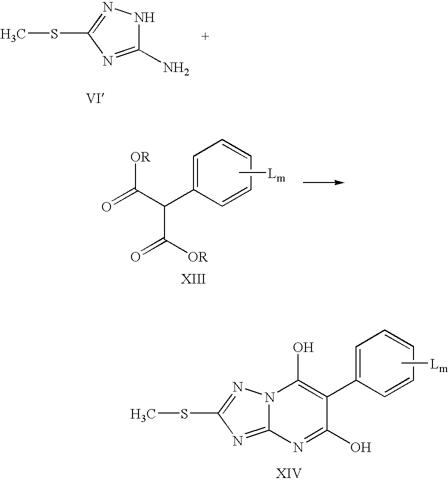

This reaction is usually carried out at temperatures of from 80° C. to 250° C., preferably from 120° C. to 180° C., without solvent or in an inert organic solvent in the presence of a base [cf. EP-A 770 615] or in the presence of acetic acid under the conditions disclosed in Adv. Het. Chem. 57, (1993), 81ff.

Suitable solvents are aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide. With particular preference, the reaction is carried out without solvent or in chlorobenzene, xylene, dimethyl sulfoxide or N-methylpyrrolidone. It is also possible to use mixtures of the solvents mentioned.

Suitable bases are, in particular, organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to using tertiary amines, such as triisopropylethylamine, tributylamine, N-methylmorpholine or N-methylpiperidine. The bases are generally employed in catalytic amounts; however, they can also be employed in equimolar amounts, in excess or, if appropriate, as solvents.

Starting materials are generally reacted with one another in equimolar amounts. In terms of yield, it may be advantageous to employ base and malonate XIII in excess, based on the triazole VI.

3-Thio-5-aminotriazole of the formula VI′ is commercially available. The phenylmalonates of the formula XIII are advantageously obtained by reacting appropriately substituted bromobenzenes with dialkyl malonates under Cu(I) catalysis [cf. Chemistry Letters, (1981), 367-370; EP-A 10 02 788].

The dihydroxytriazolopyrimidines of the formula XIV are converted into the dihalopyrimidines of the formula XV using the conditions known from WO 94/20501. The halogenating agent [Hal] used is advantageously a chlorinating agent or a brominating agent, such as phosphorus oxybromide or phosphorus oxychloride, if appropriate in the presence of a solvent.

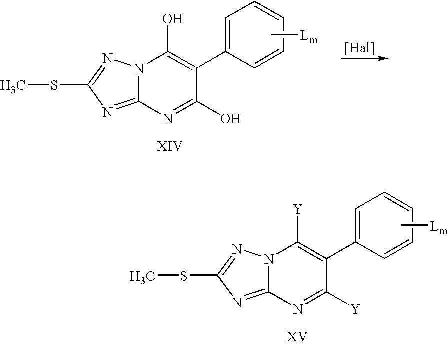

This reaction is usually carried out at from 0° C. to 150° C., preferably from 80° C. to 125° C., [cf. EP-A 770 615].

Dihalopyrimidines of the formula XV are reacted further with amines of the formula X to give compounds of the formula II.

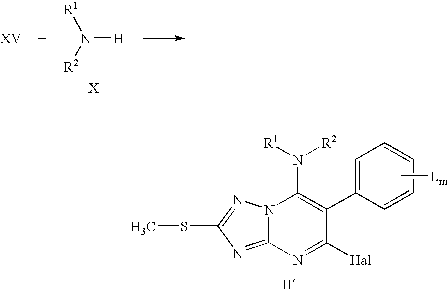

Compounds of the formula II′ are known per se from WO 02/88127.

If R¹ or R² contains haloalkyl or haloalkenyl groups, the (S)-configuration is preferred for optically active amines of the formula X.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be converted into one another during work-up for use or during application (for example under the action of light, acids or bases). Such conversions may also take place after use, for example in the treatment of plants in the treated plants or in the harmful fungus to be controlled.

In the definitions of the symbols given in the formulae above, collective terms were used which are generally representative for the following substituents:
halogen: fluorine, chlorine, bromine and iodine;
alkyl: saturated straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 10 carbon atoms (as mentioned above), where in these groups some or all of the hydrogen atoms may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6, 8 or 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkadienyl: unsaturated straight-chain or branched hydrocarbon radicals having 4, 6, 8 or 10 carbon atoms and two double bonds in any position;
haloalkenyl: unsaturated straight-chain or branched hydrocarbon radicals having 2 to 10 carbon atoms and a double bond in any position (as mentioned above), where in these groups some or all of the hydrogen atoms may be replaced by halogen atoms as mentioned above, in particular by fluorine, chlorine and bromine;
alkynyl: straight-chain or branched hydrocarbon groups having 2 to 4, 6, 8 or 10 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;
Oxyalkyleneoxy: divalent unbranched chains of 1 to 3 CH₂ groups, where both valences are attached to the skeleton via an oxygen atom, for example OCH₂O, OCH₂CH₂O and OCH₂CH₂CH₂O.
   five- or six-membered saturated, partially unsaturated or aromatic heterocycle which contains one to four heteroatoms from the group consisting of O, N and S:
5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl and 1,3,4-triazol-2-yl;
6-membered heteroaryl which contains one to three or one to four nitrogen atoms: 6-membered heteroaryl groups which, in addition to carbon atoms, may contain one to three and one to four nitrogen atoms, respectively, as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.
Alkylene: divalent unbranched chains of 3 to 5 CH₂-groups, for example CH₂, CH₂CH₂, CH₂CH₂CH₂, CH₂CH₂CH₂CH₂ and CH₂CH₂CH₂CH₂CH₂;
Oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
Oxyalkyleneoxy: divalent unbranched chains of 1 to 3 CH₂ groups, where both valences are attached to the skeleton via an oxygen atom, for example OCH₂O, OCH₂CH₂O and OCH₂CH₂CH₂O.

The scope of the present invention includes the (R)- and (S)-isomers and the racemates of compounds of the formula I having chiral centers.

With respect to the variables, the particularly preferred embodiments of the intermediates correspond to those of radicals Lₘ, R¹, R² and X of formula I.

With a view to the intended use of the triazolopyrimidines of the formula I, particular preference is given to the following meanings of the substituents, in each case on their own or in combination:

Preference is given to compounds I in which R¹ is C₁-C₄-alkyl or C₁-C₈-haloalkyl.

Particular preference is given to compounds I in which R¹ is a group B 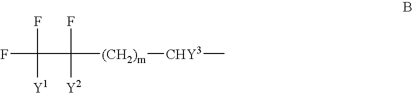
in which
Y¹ is hydrogen, fluorine or C₁-C₆-fluoroalkyl,
Y² is hydrogen or fluorine, or
   Y¹ and Y² together form a double bond;
m is 0 or 1; and
Y³ is hydrogen or methyl.

Moreover, preference is given to compounds I in which R¹ is C₃-C₆-cycloalkyl, which may be substituted by C₁-C₄-alkyl.

Especially preferred are compounds I in which R² is hydrogen.

Preference is likewise given to compounds I in which R² is methyl or ethyl.

If R¹ and/or R² contain haloalkyl or haloalkenyl groups having a center of chirality, preference is given to the (S)-isomers.

If R¹ and/or R² contain alkyl, alkenyl or alkynyl groups having a center of chirality, preference is given to the (R)-isomers.

Particular preference is furthermore given to compounds I in which R¹ and R² together with the nitrogen atom to which they are attached form a five- or six-membered ring which may be interrupted by an atom from the group consisting of O, N and S and/or may carry one or more substituents from the group consisting of halogen, C₁-C₆-alkyl, C₁-C₆-haloalkyl and oxy-C₁-C₃-alkyleneoxy or in which a nitrogen atom and an adjacent carbon atom may be linked by a C₁-C₄-alkylene chain.

Particular preference is given to compounds I in which R¹ and R² together with the nitrogen atom to which they are attached form a five- or six-membered ring which may have a double bond and which may be substituted as described above.

Especially preferred are in particular compounds I in which R¹ and R² together with the nitrogen atom to which they are attached form a piperidinyl, morpholinyl or thiomorpholinyl ring, in particular a piperidinyl ring, which is unsubstituted or substituted by one to three halogen, C₁-C₄-alkyl or C₁-C₄-haloalkyl groups, in particular a piperidinyl ring substituted by 4-methyl.

Particular preference is furthermore given to compounds I in which R¹ and R² together with the nitrogen atom to which they are attached form a pyrrolidine ring which is unsubstituted or substituted by one or two halogen, C₁-C₄-alkyl or C₁-C₄-haloalkyl groups, in particular by 2-methyl.

Preference is given to compounds I in which R³ is hydroxyl, cyano, C₁-C₄-alkoxy or NR¹R².

Preference is given to compounds I in which at least one group L is located also to the point of attachment with the triazolopyrimidine skeletons; in particular those in which m has the value 1, 2 or 3.

Preference is given to compounds I in which Lₘ is halogen, methyl, ethyl, C₁-haloalkyl, methoxy or —C(═O)-A, where A is hydrogen, hydroxyl, C₁-C₄-alkoxy, C₁-C₄-haloalkoxy, C₁-C₂-alkylamino or di-(C₁-C₂-alkyl)amino.

Moreover, particular preference is given to compounds I in which Lₘ is the group A 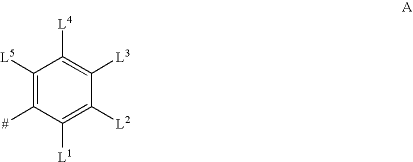
in which # is the point of attachment with the triazolopyrimidine skeleton and
L¹ is fluorine, chlorine, CH₃ or CF₃;
L²,L⁴ independently of one another are hydrogen or fluorine;
L³ is hydrogen, fluorine, chlorine, cyano, CH₃, SCH₃, SO₂CH₃, OCH₃ or COOCH₃; and
L⁵ is hydrogen, fluorine or CH₃.

Particular preference is given to compounds I in which Lₘ is one of the combinations of substituents below: 2-fluoro-6-chloro, 2,6-difluoro, 2,6-dichloro, 2-fluoro-6-methyl, 2,4,6-trifluoro, 2,6-difluoro-4-methoxy, pentafluoro, 2-methyl-4-fluoro, 2-trifluoromethyl, 2-methoxy-6-fluoro, 2-chloro, 2-fluoro, 2,4-difluoro, 2-fluoro-4-chloro, 2-chloro-4-fluoro, 2,3-difluoro, 2,5-difluoro, 2,3,4-trifluoro, 2-methyl, 2,4-dimethyl, 2-methyl-4-chloro, 2-fluoro-4-methyl, 2,6-dimethyl, 2,4,6-trimethyl, 2,6-difluoro-4-methyl, 2-trifluoromethyl-4-fluoro, 2-trifluoromethyl-5-fluoro or 2-trifluoromethyl-5-chloro.

Particular preference is given to compounds I in which X is C₁-C₄-alkyl, in particular methyl.

With a view to their use, particular preference is given to the compounds I compiled in the tables below. Moreover, the groups mentioned for a substituent in the tables are per se, independently of the combination in which they are mentioned, a particularly preferred embodiment of the substituent in question.

Table 1

Compounds of the formula I, in which X is methyl, Lₘ is 2-fluoro-6-chloro, R³ is methoxy and the combination of R¹ and R² corresponds for each compound to one row of Table A

Table 2

Compounds of the formula I, in which X is methyl, Lₘ is 2,6-difluoro, R³ is methoxy and the combination of R¹ and R² corresponds for each compound to one row of Table A

Table 3

Compounds of the formula I, in which X is methyl, Lₘ is 2,6-dichloro, R³ is methoxy and the combination of R¹ and R² corresponds for each compound to one row of Table A

Table 4

Compounds of the formula I, in which X is methyl, Lₘ is 2-fluoro-6-methyl, R³ is methoxy and the combination of R¹ and R² corresponds for each compound to one row of Table A

Table 5

Compounds of the formula I, in which X is methyl, Lₘ is 2,4,6-trifluoro, R³ is methoxy and the combination of R¹ and R² corresponds for each compound to one row of Table A

Table 6

Compounds of the formula I, in which X is methyl, Lₘ is 2,6-difluoro-4-methoxy, R³ is methoxy and the combination of R¹ and R² corresponds for each compound to one row of Table A

Table 7

Compounds of the formula I, in which X is methyl, Lₘ is pentafluoro, R³ is methoxy and the combination of R¹ and R² corresponds for each compound to one row of Table A

Table 8

Compounds of the formula I, in which X is methyl, Lₘ is 2-methyl-4-fluoro, R³ is methoxy and the combination of R¹ and R² corresponds for each compound to one row of Table A

Table 9

Compounds of the formula I, in which X is methyl, Lₘ is 2-trifluoromethyl, R³ is methoxy and the combination of R¹ and R² corresponds for each compound to one row of Table A

Table 10

Compounds of the formula I, in which X is methyl, Lₘ is 2-methoxy-6-fluoro, R³ is methoxy and the combination of R¹ and R² corresponds for each compound to one row of Table A

Table 11

Compounds of the formula I, in which X is methyl, Lₘ is 2-chloro, R³ is methoxy and the combination of R¹ and R² corresponds for each compound to one row of Table A

Table 12

Compounds of the formula I, in which X is methyl, Lₘ is 2-fluoro, R³ is methoxy and the combination of R¹ and R² corresponds for each compound to one row of Table A

Table 13

Compounds of the formula I, in which X is methyl, Lₘ is 2,4-difluoro, R³ is methoxy and the combination of R¹ and R² corresponds for each compound to one row of Table A

Table 14

Compounds of the formula I, in which X is methyl, Lₘ is 2-fluoro-4-chloro, R³ is methoxy and the combination of R¹ and R² corresponds for each compound to one row of Table A

Table 15

Compounds of the formula I, in which X is methyl, Lₘ is 2-chloro-4-fluoro, R³ is methoxy and the combination of R¹ and R² corresponds for each compound to one row of Table A

Table 16

Compounds of the formula I, in which X is methyl, Lₘ is 2,3-difluoro, R³ is methoxy and the combination of R¹ and R² corresponds for each compound to one row of Table A

Table 17

Compounds of the formula I, in which X is methyl, Lₘ is 2,5-difluoro, R³ is methoxy and the combination of R¹ and R² corresponds for each compound to one row of Table A

Table 18

Compounds of the formula I, in which X is methyl, Lₘ is 2,3,4-trifluoro, R³ is methoxy and the combination of R¹ and R² corresponds for each compound to one row of Table A

Table 19

Compounds of the formula I, in which X is methyl, Lₘ is 2-methyl, R³ is methoxy and the combination of R¹ and R² corresponds for each compound to one row of Table A

Table 20

Compounds of the formula I, in which X is methyl, Lₘ is 2,4-dimethyl, R³ is methoxy and the combination of R¹ and R² corresponds for each compound to one row of Table A

Table 21

Compounds of the formula I, in which X is methyl, Lₘ is 2-methyl-4-chloro, R³ is methoxy and the combination of R¹ and R² corresponds for each compound to one row of Table A

Table 22

Compounds of the formula I, in which X is methyl, Lₘ is 2-fluoro-4-methyl, R³ is methoxy and the combination of R¹ and R² corresponds for each compound to one row of Table A

Table 23

Compounds of the formula I, in which X is methyl, Lₘ is 2,6-dimethyl, R³ is methoxy and the combination of R¹ and R² corresponds for each compound to one row of Table A

Table 24

Compounds of the formula I, in which X is methyl, Lₘ is 2,4,6-trimethyl, R³ is methoxy and the combination of R¹ and R² corresponds for each compound to one row of Table A

Table 25

Compounds of the formula I, in which X is methyl, Lₘ is 2,6-difluoro-4-cyano, R³ is methoxy and the combination of R¹ and R² corresponds for each compound to one row of Table A

Table 26

Compounds of the formula I, in which X is methyl, Lₘ is 2,6-difluoro-4-methyl, R³ is methoxy and the combination of R¹ and R² corresponds for each compound to one row of Table A

Table 27

Compounds of the formula I, in which X is methyl, Lₘ is 2,6-difluoro-4-methoxycarbonyl, R³ is methoxy and the combination of R¹ and R² corresponds for each compound to one row of Table A

Table 28

Compounds of the formula I, in which X is methyl, Lₘ is 2-trifluoromethyl-4-fluoro, R³ is methoxy and the combination of R¹ and R² corresponds for each compound to one row of Table A

Table 29

Compounds of the formula I, in which X is methyl, Lₘ is 2-trifluoromethyl-5-fluoro, R³ is methoxy and the combination of R¹ and R² corresponds for each compound to one row of Table A

Table 30

Compounds of the formula I, in which X is methyl, Lₘ is 2-trifluoromethyl-5-chloro, R³ is methoxy and the combination of R¹ and R² corresponds for each compound to one row of Table A

Table 31

Compounds of the formula I, in which X is methyl, Lₘ is 2-fluoro-6-chloro, R³ is cyano and the combination of R¹ and R² corresponds for each compound to one row of Table A

Table 32

Compounds of the formula I, in which X is methyl, Lₘ is 2,6-difluoro, R³ is cyano and the combination of R¹ and R² corresponds for each compound to one row of Table A

Table 33

Compounds of the formula I, in which X is methyl, Lₘ is 2,6-dichloro, R³ is cyano and the combination of R¹ and R² corresponds for each compound to one row of Table A

Table 34

Compounds of the formula I, in which X is methyl, Lₘ is 2-fluoro-6-methyl, R³ is cyano and the combination of R¹ and R² corresponds for each compound to one row of Table A

Table 35

Compounds of the formula I, in which X is methyl, Lₘ is 2,4,6-trifluoro, R³ is cyano and the combination of R¹ and R² corresponds for each compound to one row of Table A

Table 36

Compounds of the formula I, in which X is methyl, Lₘ is 2,6-difluoro-4-methoxy, R³ is cyano and the combination of R¹ and R² corresponds for each compound to one row of Table A

Table 37

Compounds of the formula I, in which X is methyl, Lₘ is pentafluoro, R³ is cyano and the combination of R¹ and R² corresponds for each compound to one row of Table A

Table 38

Compounds of the formula I, in which X is methyl, Lₘ is 2-methyl-4-fluoro, R³ is cyano and the combination of R¹ and R² corresponds for each compound to one row of Table A

Table 39

Compounds of the formula I, in which X is methyl, Lₘ is 2-trifluoromethyl, R³ is cyano and the combination of R¹ and R² corresponds for each compound to one row of Table A

Table 40

Compounds of the formula I, in which X is methyl, Lₘ is 2-methoxy-6-fluoro, R³ is cyano and the combination of R¹ and R² corresponds for each compound to one row of Table A

Table 41

Compounds of the formula I, in which X is methyl, Lₘ is 2-chloro, R³ is cyano and the combination of R¹ and R² corresponds for each compound to one row of Table A

Table 42

Compounds of the formula I, in which X is methyl, Lₘ is 2-fluoro, R³ is cyano and the combination of R¹ and R² corresponds for each compound to one row of Table A

Table 43

Compounds of the formula I, in which X is methyl, Lₘ is 2,4-difluoro, R³ is cyano and the combination of R¹ and R² corresponds for each compound to one row of Table A

Table 44

Compounds of the formula I, in which X is methyl, Lₘ is 2-fluoro-4-chloro, R³ is cyano and the combination of R¹ and R² corresponds for each compound to one row of Table A

Table 45

Compounds of the formula I, in which X is methyl, Lₘ is 2-chloro-4-fluoro, R³ is cyano and the combination of R¹ and R² corresponds for each compound to one row of Table A

Table 46

Compounds of the formula I, in which X is methyl, Lₘ is 2,3-difluoro, R³ is cyano and the combination of R¹ and R² corresponds for each compound to one row of Table A

Table 47

Compounds of the formula I, in which X is methyl, Lₘ is 2,5-difluoro, R³ is cyano and the combination of R¹ and R² corresponds for each compound to one row of Table A

Table 48

Compounds of the formula I, in which X is methyl, Lₘ is 2,3,4-trifluoro, R³ is cyano and the combination of R¹ and R² corresponds for each compound to one row of Table A

Table 49

Compounds of the formula I, in which X is methyl, Lₘ is 2-methyl, R³ is cyano and the combination of R¹ and R² corresponds for each compound to one row of Table A

Table 50

Compounds of the formula I, in which X is methyl, Lₘ is 2,4-dimethyl, R³ is cyano and the combination of R¹ and R² corresponds for each compound to one row of Table A

Table 51

Compounds of the formula I, in which X is methyl, Lₘ is 2-methyl-4-chloro, R³ is cyano and the combination of R¹ and R² corresponds for each compound to one row of Table A

Table 52

Compounds of the formula I, in which X is methyl, Lₘ is 2-fluoro-4-methyl, R³ is cyano and the combination of R¹ and R² corresponds for each compound to one row of Table A

Table 53

Compounds of the formula I, in which X is methyl, Lₘ is 2,6-dimethyl, R³ is cyano and the combination of R¹ and R² corresponds for each compound to one row of Table A

Table 54

Compounds of the formula I, in which X is methyl, Lₘ is 2,4,6-trimethyl, R³ is cyano and the combination of R¹ and R² corresponds for each compound to one row of Table A

Table 55

Compounds of the formula I, in which X is methyl, Lₘ is 2,6-difluoro-4-cyano, R³ is cyano and the combination of R¹ and R² corresponds for each compound to one row of Table A

Table 56

Compounds of the formula I, in which X is methyl, Lₘ is 2,6-difluoro-4-methyl, R³ is cyano and the combination of R¹ and R² corresponds for each compound to one row of Table A

Table 57

Compounds of the formula I, in which X is methyl, Lₘ is 2,6-difluoro-4-methoxycarbonyl, R³ is cyano and the combination of R¹ and R² corresponds for each compound to one row of Table A

Table 58

Compounds of the formula I, in which X is methyl, Lₘ is 2-trifluoromethyl-4-fluoro, R³ is cyano and the combination of R¹ and R² corresponds for each compound to one row of Table A

Table 59

Compounds of the formula I, in which X is methyl, Lₘ is 2-trifluoromethyl-5-fluoro, R³ is cyano and the combination of R¹ and R² corresponds for each compound to one row of Table A

Table 60

Compounds of the formula I, in which X is methyl, Lₘ is 2-trifluoromethyl-5-chloro, R³ is cyano and the combination of R¹ and R² corresponds for each compound to one row of Table A

Table 61

Compounds of the formula I, in which X is methyl, Lₘ is 2-fluoro-6-chloro, R³ is dimethylamino and the combination of R¹ and R² corresponds for each compound to one row of Table A

Table 62

Compounds of the formula I, in which X is methyl, Lₘ is 2,6-difluoro, R³ is dimethylamino and the combination of R¹ and R² corresponds for each compound to one row of Table A

Table 63

Compounds of the formula I, in which X is methyl, Lₘ is 2,6-dichloro, R³ is dimethylamino and the combination of R¹ and R² corresponds for each compound to one row of Table A

Table 64

Compounds of the formula I, in which X is methyl, Lₘ is 2-fluoro-6-methyl, R³ is dimethylamino and the combination of R¹ and R² corresponds for each compound to one row of Table A

Table 65

Compounds of the formula I, in which X is methyl, Lₘ is 2,4,6-trifluoro, R³ is dimethylamino and the combination of R¹ and R² corresponds for each compound to one row of Table A

Table 66

Compounds of the formula I, in which X is methyl, Lₘ is 2,6-difluoro-4-methoxy, R³ is dimethylamino and the combination of R¹ and R² corresponds for each compound to one row of Table A

Table 67

Compounds of the formula I, in which X is methyl, Lₘ is pentafluoro, R³ is dimethylamino and the combination of R¹ and R² corresponds for each compound to one row of Table A

Table 68

Compounds of the formula I, in which X is methyl, Lₘ is 2-methyl-4-fluoro, R³ is dimethylamino and the combination of R¹ and R² corresponds for each compound to one row of Table A

Table 69

Compounds of the formula I, in which X is methyl, Lₘ is 2-trifluoromethyl, R³ is dimethylamino and the combination of R¹ and R² corresponds for each compound to one row of Table A

Table 70

Compounds of the formula I, in which X is methyl, Lₘ is 2-methoxy-6-fluoro, R³ is dimethylamino and the combination of R¹ and R² corresponds for each compound to one row of Table A

Table 71

Compounds of the formula I, in which X is methyl, Lₘ is 2-chloro, R³ is dimethylamino and the combination of R¹ and R² corresponds for each compound to one row of Table A

Table 72

Compounds of the formula I, in which X is methyl, Lₘ is 2-fluoro, R³ is dimethylamino and the combination of R¹ and R² corresponds for each compound to one row of Table A

Table 73

Compounds of the formula I, in which X is methyl, Lₘ is 2,4-difluoro, R³ is dimethylamino and the combination of R¹ and R² corresponds for each compound to one row of Table A

Table 74

Compounds of the formula I, in which X is methyl, Lₘ is 2-fluoro-4-chloro, R³ is dimethylamino and the combination of R¹ and R² corresponds for each compound to one row of Table A

Table 75

Compounds of the formula I, in which X is methyl, Lₘ is 2-chloro-4-fluoro, R³ is dimethylamino and the combination of R¹ and R² corresponds for each compound to one row of Table A

Table 76

Compounds of the formula I, in which X is methyl, Lₘ is 2,3-difluoro, R³ is dimethylamino and the combination of R¹ and R² corresponds for each compound to one row of Table A

Table 77

Compounds of the formula I, in which X is methyl, Lₘ is 2,5-difluoro, R³ is dimethylamino and the combination of R¹ and R² corresponds for each compound to one row of Table A

Table 78

Compounds of the formula I, in which X is methyl, Lₘ is 2,3,4-trifluoro, R³ is dimethylamino and the combination of R¹ and R² corresponds for each compound to one row of Table A

Table 79

Compounds of the formula I, in which X is methyl, Lₘ is 2-methyl, R³ is dimethylamino and the combination of R¹ and R² corresponds for each compound to one row of Table A

Table 80

Compounds of the formula I, in which X is methyl, Lₘ is 2,4-dimethyl, R³ is dimethylamino and the combination of R¹ and R² corresponds for each compound to one row of Table A

Table 81

Compounds of the formula I, in which X is methyl, Lₘ is 2-methyl-4-chloro, R³ is dimethylamino and the combination of R¹ and R² corresponds for each compound to one row of Table A

Table 82

Compounds of the formula I, in which X is methyl, Lₘ is 2-fluoro-4-methyl, R³ is dimethylamino and the combination of R¹ and R² corresponds for each compound to one row of Table A

Table 83

Compounds of the formula I, in which X is methyl, Lₘ is 2,6-dimethyl, R³ is dimethylamino and the combination of R¹ and R² corresponds for each compound to one row of Table A

Table 84

Compounds of the formula I, in which X is methyl, Lₘ is 2,4,6-trimethyl, R³ is dimethylamino and the combination of R¹ and R² corresponds for each compound to one row of Table A

Table 85

Compounds of the formula I, in which X is methyl, Lₘ is 2,6-difluoro-4-cyano, R³ is dimethylamino and the combination of R¹ and R² corresponds for each compound to one row of Table A

Table 86

Compounds of the formula I, in which X is methyl, Lₘ is 2,6-difluoro-4-methyl, R³ is dimethylamino and the combination of R¹ and R² corresponds for each compound to one row of Table A

Table 87

Compounds of the formula I, in which X is methyl, Lₘ is 2,6-difluoro-4-methoxycarbonyl, R³ is dimethylamino and the combination of R¹ and R² corresponds for each compound to one row of Table A

Table 88

Compounds of the formula I, in which X is methyl, Lₘ is 2-trifluoromethyl-4-fluoro, R³ is dimethylamino and the combination of R¹ and R² corresponds for each compound to one row of Table A

Table 89

Compounds of the formula I, in which X is methyl, Lₘ is 2-trifluoromethyl-5-fluoro, R³ is dimethylamino and the combination of R¹ and R² corresponds for each compound to one row of Table A

Table 90

Compounds of the formula I, in which X is methyl, Lₘ is 2-trifluoromethyl-5-chloro, R³ is dimethylamino and the combination of R¹ and R² corresponds for each compound to one row of Table A

Table 91

Compounds of the formula I, in which X is methyl, Lₘ is 2-fluoro-6-chloro, R³ is trifluoromethoxy and the combination of R¹ and R² corresponds for each compound to one row of Table A

Table 92

Compounds of the formula I, in which X is methyl, Lₘ is 2,6-difluoro, R³ is trifluoromethoxy and the combination of R¹ and R² corresponds for each compound to one row of Table A

Table 93

Compounds of the formula I, in which X is methyl, Lₘ is 2,6-dichloro, R³ is trifluoromethoxy and the combination of R¹ and R² corresponds for each compound to one row of Table A

Table 94

Compounds of the formula I, in which X is methyl, Lₘ is 2-fluoro-6-methyl, R³ is trifluoromethoxy and the combination of R¹ and R² corresponds for each compound to one row of Table A

Table 95

Compounds of the formula I, in which X is methyl, Lₘ is 2,4,6-trifluoro, R³ is trifluoromethoxy and the combination of R¹ and R² corresponds for each compound to one row of Table A

Table 96

Compounds of the formula I, in which X is methyl, Lₘ is 2,6-difluoro-4-methoxy, R³ is trifluoromethoxy and the combination of R¹ and R² corresponds for each compound to one row of Table A

Table 97

Compounds of the formula I, in which X is methyl, Lₘ is pentafluoro, R³ is trifluoromethoxy and the combination of R¹ and R² corresponds for each compound to one row of Table A

Table 98

Compounds of the formula I, in which X is methyl, Lₘ is 2-methyl-4-fluoro, R³ is trifluoromethoxy and the combination of R¹ and R² corresponds for each compound to one row of Table A

Table 99

Compounds of the formula I, in which X is methyl, Lₘ is 2-trifluoromethyl, R³ is trifluoromethoxy and the combination of R¹ and R² corresponds for each compound to one row of Table A

Table 100

Compounds of the formula I, in which X is methyl, Lₘ is 2-methoxy-6-fluoro, R³ is trifluoromethoxy and the combination of R¹ and R² corresponds for each compound to one row of Table A

Table 101

Compounds of the formula I, in which X is methyl, Lₘ is 2-chloro, R³ is trifluoromethoxy and the combination of R¹ and R² corresponds for each compound to one row of Table A

Table 102

Compounds of the formula I, in which X is methyl, Lₘ is 2-fluoro, R³ is trifluoromethoxy and the combination of R¹ and R² corresponds for each compound to one row of Table A

Table 103

Compounds of the formula I, in which X is methyl, Lₘ is 2,4-difluoro, R³ is trifluoromethoxy and the combination of R¹ and R² corresponds for each compound to one row of Table A

Table 104

Compounds of the formula I, in which X is methyl, Lₘ is 2-fluoro-4-chloro, R³ is trifluoromethoxy and the combination of R¹ and R² corresponds for each compound to one row of Table A

Table 105

Compounds of the formula I, in which X is methyl, Lₘ is 2-chloro-4-fluoro, R³ is trifluoromethoxy and the combination of R¹ and R² corresponds for each compound to one row of Table A

Table 106

Compounds of the formula I, in which X is methyl, Lₘ is 2,3-difluoro, R³ is trifluoromethoxy and the combination of R¹ and R² corresponds for each compound to one row of Table A

Table 107

Compounds of the formula I, in which X is methyl, Lₘ is 2,5-difluoro, R³ is trifluoromethoxy and the combination of R¹ and R² corresponds for each compound to one row of Table A

Table 108

Compounds of the formula I, in which X is methyl, Lₘ is 2,3,4-trifluoro, R³ is trifluoromethoxy and the combination of R¹ and R² corresponds for each compound to one row of Table A

Table 109

Compounds of the formula I, in which X is methyl, Lₘ is 2-methyl, R³ is trifluoromethoxy and the combination of R¹ and R² corresponds for each compound to one row of Table A

Table 110

Compounds of the formula I, in which X is methyl, Lₘ is 2,4-dimethyl, R³ is trifluoromethoxy and the combination of R¹ and R² corresponds for each compound to one row of Table A

Table 111

Compounds of the formula I, in which X is methyl, Lₘ is 2-methyl-4-chloro, R³ is trifluoromethoxy and the combination of R¹ and R² corresponds for each compound to one row of Table A

Table 112

Compounds of the formula I, in which X is methyl, Lₘ is 2-fluoro-4-methyl, R³ is trifluoromethoxy and the combination of R¹ and R² corresponds for each compound to one row of Table A

Table 113

Compounds of the formula I, in which X is methyl, Lₘ is 2,6-dimethyl, R³ is trifluoromethoxy and the combination of R¹ and R² corresponds for each compound to one row of Table A

Table 114

Compounds of the formula I, in which X is methyl, Lₘ is 2,4,6-trimethyl, R³ is trifluoromethoxy and the combination of R¹ and R² corresponds for each compound to one row of Table A

Table 115

Compounds of the formula I, in which X is methyl, Lₘ is 2,6-difluoro-4-cyano, R³ is trifluoromethoxy and the combination of R¹ and R² corresponds for each compound to one row of Table A

Table 116

Compounds of the formula I, in which X is methyl, Lₘ is 2,6-difluoro-4-methyl, R³ is trifluoromethoxy and the combination of R¹ and R² corresponds for each compound to one row of Table A

Table 117

Compounds of the formula I, in which X is methyl, Lₘ is 2,6-difluoro-4-methoxycarbonyl, R³ is trifluoromethoxy and the combination of R¹ and R² corresponds for each compound to one row of Table A

Table 118

Compounds of the formula I, in which X is methyl, Lₘ is 2-trifluoromethyl-4-fluoro, R³ is trifluoromethoxy and the combination of R¹ and R² corresponds for each compound to one row of Table A

Table 119

Compounds of the formula I, in which X is methyl, Lₘ is 2-trifluoromethyl-5-fluoro, R³ is trifluoromethoxy and the combination of R¹ and R² corresponds for each compound to one row of Table A

Table 120

Compounds of the formula I, in which X is methyl, Lₘ is 2-trifluoromethyl-5-chloro, R³ is trifluoromethoxy and the combination of R¹ and R² corresponds for each compound to one row of Table A

TABLE A

| No. | R¹ | R² |
|---|---|---|
| A-1 | H | H |
| A-2 | CH₂CH₃ | H |
| A-3 | CH₂CH₃ | CH₃ |
| A-4 | CH₂CH₃ | CH₂CH₃ |
| A-5 | CH₂CF₃ | H |
| A-6 | CH₂CF₃ | CH₃ |
| A-7 | CH₂CF₃ | CH₂CH₃ |
| A-8 | CH₂CCl₃ | H |
| A-9 | CH₂CCl₃ | CH₃ |
| A-10 | CH₂CCl₃ | CH₂CH₃ |
| A-11 | CH₂CH₂CH₃ | H |
| A-12 | CH₂CH₂CH₃ | CH₃ |
| A-13 | CH₂CH₂CH₃ | CH₂CH₃ |
| A-14 | CH₂CH₂CH₃ | CH₂CH₂CH₃ |
| A-15 | CH(CH₃)₂ | H |
| A-16 | CH(CH₃)₂ | CH₃ |
| A-17 | CH(CH₃)₂ | CH₂CH₃ |
| A-18 | (±) CH(CH₃)—CH₂CH₃ | H |
| A-19 | (±) CH(CH₃)—CH₂CH₃ | CH₃ |
| A-20 | (±) CH(CH₃)—CH₂CH₃ | CH₂CH₃ |
| A-21 | (s) CH(CH₃)—CH₂CH₃ | H |
| A-22 | (S) CH(CH₃)—CH₂CH₃ | CH₃ |
| A-23 | (S) CH(CH₃)—CH₂CH₃ | CH₂CH₃ |
| A-24 | (R) CH(CH₃)—CH₂CH₃ | H |
| A-25 | (R) CH(CH₃)—CH₂CH₃ | CH₃ |
| A-26 | (R) CH(CH₃)—CH₂CH₃ | CH₂CH₃ |
| A-27 | (±) CH(CH₃)—CH(CH₃)₂ | H |
| A-28 | (±) CH(CH₃)—CH(CH₃)₂ | CH₃ |
| A-29 | (±) CH(CH₃)—CH(CH₃)₂ | CH₂CH₃ |
| A-30 | (S) CH(CH₃)—CH(CH₃)₂ | H |
| A-31 | (S) CH(CH₃)—CH(CH₃)₂ | CH₃ |
| A-32 | (S) CH(CH₃)—CH(CH₃)₂ | CH₂CH₃ |
| A-33 | (R) CH(CH₃)—CH(CH₃)₂ | H |
| A-34 | (R) CH(CH₃)—CH(CH₃)₂ | CH₃ |
| A-35 | (R) CH(CH₃)—CH(CH₃)₂ | CH₂CH₃ |
| A-36 | (±) CH(CH₃)—C(CH₃)₃ | H |
| A-37 | (±) CH(CH₃)—C(CH₃)₃ | CH₃ |
| A-38 | (±) CH(CH₃)—C(CH₃)₃ | CH₂CH₃ |
| A-39 | (S) CH(CH₃)—C(CH₃)₃ | H |
| A-40 | (S) CH(CH₃)—C(CH₃)₃ | CH₃ |
| A-41 | (S) CH(CH₃)—C(CH₃)₃ | CH₂CH₃ |
| A-42 | (R) CH(CH₃)—C(CH₃)₃ | H |
| A-43 | (R) CH(CH₃)—C(CH₃)₃ | CH₃ |
| A-44 | (R) CH(CH₃)—C(CH₃)₃ | CH₂CH₃ |
| A-45 | (±) CH(CH₃)—CF₃ | H |
| A-46 | (±) CH(CH₃)—CF₃ | CH₃ |
| A-47 | (±) CH(CH₃)—CF₃ | CH₂CH₃ |
| A-48 | (S) CH(CH₃)—CF₃ | H |
| A-49 | (S) CH(CH₃)—CF₃ | CH₃ |
| A-50 | (S) CH(CH₃)—CF₃ | CH₂CH₃ |
| A-51 | (R) CH(CH₃)—CF₃ | H |
| A-52 | (R) CH(CH₃)—CF₃ | CH₃ |
| A-53 | (R) CH(CH₃)—CF₃ | CH₂CH₃ |
| A-54 | (±) CH(CH₃)—CCl₃ | H |
| A-55 | (±) CH(CH₃)—CCl₃ | CH₃ |
| A-56 | (±) CH(CH₃)—CCl₃ | CH₂CH₃ |
| A-57 | (S) CH(CH₃)—CCl₃ | H |
| A-58 | (S) CH(CH₃)—CCl₃ | CH₃ |
| A-59 | (S) CH(CH₃)—CCl₃ | CH₂CH₃ |
| A-60 | (R) CH(CH₃)—CCl₃ | H |
| A-61 | (R) CH(CH₃)—CCl₃ | CH₃ |
| A-62 | (R) CH(CH₃)—CCl₃ | CH₂CH₃ |
| A-63 | CH₂CF₂CF₃ | H |
| A-64 | CH₂CF₂CF₃ | CH₃ |
| A-65 | CH₂CF₂CF₃ | CH₂CH₃ |
| A-66 | CH₂(CF₂)₂CF₃ | H |
| A-67 | CH₂(CF₂)₂CF₃ | CH₃ |
| A-68 | CH₂(CF₂)₂CF₃ | CH₂CH₃ |
| A-69 | CH₂C(CH₃)═CH₂ | H |
| A-70 | CH₂C(CH₃)═CH₂ | CH₃ |
| A-71 | CH₂C(CH₃)═CH₂ | CH₂CH₃ |
| A-72 | CH₂CH═CH₂ | H |
| A-73 | CH₂CH═CH₂ | CH₃ |
| A-74 | CH₂CH═CH₂ | CH₂CH₃ |
| A-75 | CH(CH₃)CH═CH₂ | H |
| A-76 | CH(CH₃)CH═CH₂ | CH₃ |
| A-77 | CH(CH₃)CH═CH₂ | CH₂CH₃ |
| A-78 | CH(CH₃)C(CH₃)═CH₂ | H |
| A-79 | CH(CH₃)C(CH₃)═CH₂ | CH₃ |
| A-80 | CH(CH₃)C(CH₃)═CH₂ | CH₂CH₃ |
| A-81 | Cyclopentyl | H |
| A-82 | Cyclopentyl | CH₃ |
| A-83 | Cyclopentyl | CH₂CH₃ |
| A-84 | Cyclohexyl | H |
| A-85 | Cyclohexyl | CH₃ |
| A-86 | Cyclohexyl | CH₂CH₃ |

| | |
|---|---|
| A-87 | —(CH₂)₂CH═CHCH₂— |
| A-88 | —(CH₂)₂C(CH₃)═CHCH₂— |
| A-89 | —(CH₂)₂CH(CH₃)(CH₂)₂— |
| A-90 | —(CH₂)₂CHF(CH₂)₂— |
| A-91 | —(CH₂)₃CHFCH₂— |
| A-92 | —(CH₂)₂CH(CF₃)(CH₂)₂— |
| A-93 | —(CH₂)₂O(CH₂)₂— |
| A-94 | —(CH₂)₂S(CH₂)₂— |
| A-95 | —(CH₂)₅— |
| A-96 | —(CH₂)₄— |
| A-97 | —CH₂CH═CHCH₂— |
| A-98 | —CH(CH₃)(CH₂)₃— |
| A-99 | —CH₂CH(CH₃)(CH₂)₂— |

The compounds I are suitable as fungicides. They are distinguished through an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, maize, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 1 g, preferably 0.01 to 0.05 g, per kilogram of seed are generally necessary.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

A Water-Soluble Concentrates (SL)

10 parts by weight of a compound according to the invention are dissolved in water or in a water-soluble solvent. As an alternative, wetters or other auxiliaries are added. The active compound dissolves upon dilution with water.

B Dispersible Concentrates (DC)

20 parts by weight of a compound according to the invention are dissolved in cyclohexanone with addition of a dispersant, for example polyvinylpyrrolidone. Dilution with water gives a dispersion.

C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion.

D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifier (Ultraturax) and made into a homogeneous emulsion. Dilution with water gives an emulsion.

E Suspensions (SC, OD)

In an agitated ball mill, 20 parts by weight of a compound according to the invention are comminuted with addition of dispersant, wetters and water or an organic solvent to give a fine active compound suspension. Dilution with water gives a stable suspension of the active compound.

F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

G Water-Dispersible Powders and Water-Soluble Powders (WP, SP)

75 parts by weight of a compound according to the invention are ground in a rotor-stator mill with addition of dispersant, wetters and silica gel. Dilution with water gives a stable dispersion or solution with the active compound.

2. Products to be applied undiluted

H Dustable Powders (DP)

5 parts by weight of a compound according to the invention are ground finely and mixed intimately with 95% of finely divided kaolin. This gives a dust.

I Granules (GR, FG, GG, MG)

0.5 part by weight of a compound according to the invention is ground finely and associated with 95.5% carriers. Current methods are extrusion, spray-drying or fluidized bed. This gives granules to be applied undiluted.

J ULV Solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dust compositions, for broadcasting, or granules, by means of spraying, atomizing, dusting, broadcasting or pouring. The use forms depend entirely on the intended purposes; it is intended to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates suitable for dilution with water and composed of active substance, wetter, tackifier, dispersant or emulsifier and, if appropriate, solvent or oil.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), where it is possible to apply formulations comprising over 95% by weight of active compound, or even to apply the active compound without additives.

Oils of various type, wetters, adjuvants, herbicides, fungicides, other pesticides, or bactericides may be added to the active compounds, if appropriate just immediately prior to use (tank mix). These agents can be admixed with the agents according to the invention in a weight ratio of 1:10 to 10:1.

The preparations according to the invention can, in the application form as fungicides, also be present together with other active compounds, for example with herbicides, insecticides, growth regulators, fungicides or also with fertilizers. On mixing the compounds I or the preparations comprising them in the application form as fungicides with other fungicides, in many cases an expansion of the fungicidal spectrum of activity is obtained.

The following lists of fungicides, with which the compounds according to the invention can be used in conjunction, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidine, such as pyrimethanil, mepanipyrim or cyrodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, toloclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described again in the following synthesis examples can be used to prepare further compounds I by appropriate modification of the starting compounds.

### Example 1

### Preparation of 5-cyano-6-(2,4,6-trifluorophenyl)-7-(4-methylpiperidin-1-yl)-2-thiomethyl-[1,2,4]-triazolo[1,5-a]pyrimidine

A mixture of 0.1 mol of 5-chloro-6-(2,4,6-trifluorophenyl)-7-(4-methylpiperidin-1-yl)-2-thiomethyl[1,2,4]-triazolo[1,5-a]pyrimidine (WO 02/088127) and 0.25 mol of tetraethylammonium cyanide in 750 ml of dimethylformamide (DMF) was stirred at 20-25° C. for about 16 hours. Water was added, the mixture was extracted with methyl tert-butyl ether (MTBE) and the phases were then separated, after which the organic phase was washed with water and dried. Removal of the solvent by distillation was followed by chromatography of the residue on silica gel. This gave 4.21 g of the title compound of m.p. 212° C.

### Example 2

### Preparation of 5-methoxy-6-(2,4,6-trifluorophenyl)-7-(4-methylpiperidin-1-yl)-2-thiomethyl-[1,2,4]-triazolo[1,5-a]pyrimidine

At about 20-25° C., a 30% strength sodium methoxide solution (71.5 mmol) was added to a solution of 65 mmol of 5-chloro-6-(2,4,6-trifluorophenyl)-7-(4-methylpiperidin-1-yl)-2-t hiomethyl-[1,2,4]-triazolo[1,5-a]pyrimidine (WO 02/088127) in 400 ml of anhydrous methanol. And the mixture was then stirred at this temperature for about 16 hours. Methanol was distilled off and the residue was taken up in dichloromethane. The organic phase was washed with water and, after drying, the solvent was removed. The residue gave, after silica gel chromatography, 4.02 g of the title compound of m.p. 185° C.

### Example 3

### Preparation of 5-methyl-6-(2,4,6-trifluorophenyl)-7-(4-methylpiperidin-1-yl)-2-thiomethyl-[1,2,4]-triazolo[1,5-a]pyrimidine

A mixture of 20 ml of diethyl malonate and 0.27 g of sodium hydride as a 50% strength dispersion in mineral oil in 50 ml of acetonitrile was stirred at 20-25° C. for about 2 hours, 4.71 mmol of 5-chloro-6-(2,4,6-trifluorophenyl)-7-(4-methylpiperidin-1-yl)-2-thiomethyl-[1,2,4]-triazolo-[1,5-a]-pyrimidine (WO 02/088127) was then added and the mixture was stirred at about 60° C. for about 20 hours. 50 ml of an aqueous ammonium chloride solution were added and the mixture was then acidified with dilute HCl solution. The reaction mixture was extracted with methyl tert-butyl ether (MTBE) and, after phase separation, the organic phase was washed with water and dried. Removal of the solvent by distillation was followed by chromatography of the residue on silica gel.

The resulting product was taken up in dilute HCl solution, and the solution was kept at 80° C. for about 24 hours. After cooling, the pH was adjusted to 5 using aqueous NaOH solution and the solution was extracted with methyl tert-butyl ether (MTBE). The combined organic phases were dried and freed from the solvent. Silica gel chromatography gave 0.73 g of the title compound of m.p. 149° C.

### Examples of the Activity in Harmful Fungi

The fungicidal action of the compounds of the formula I was demonstrated by the following experiment:

The active compounds were formulated as a stock solution prepared using 0.25% by weight of active compound in acetone of DMSO. 1% by weight of the emulsifier Uniperol® EL (wetting agent having emulsifying and dispersant action based on ethoxylated alkylphenols) was added to this solution, and the solution was diluted with water to give the desired concentration.

### Use Example—Activity Against Early Blight of Tomato Caused by Alternaria solani, Protective Application

Leaves of potted plants of the tomato cultivar: “Large Fruited St. Pierre” were sprayed to runoff point with an aqueous suspension having the concentration of active compound stated below. The next day, the leaves were infected with an aqueous spore suspension of Alternaria solani in a 2% biomalt solution having a density of 0.17×10⁶ spores/ml. The plants were then placed in a water-vapor-saturated chamber at 20-22° C. After 5 days, the blight on the untreated but infected control plants had developed to such an extent that the infection could be determined visually in %.

In this test, the plants which had been treated with 250 ppm of the active compound from Example 3 showed 20% infection, whereas the untreated plants were 100% infected.

1. A 2-substituted triazolopyrimidine of the formula I 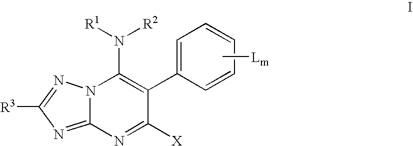
in which the substituents are as defined below:
L independently of one another are halogen, cyano, nitro, C₁-C₆-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, —C(═O)-A or S(═O)ₚA′;
A is hydrogen, hydroxyl, C₁-C₈-alkyl, C₂-C₈-alkenyl, C₁-C₈-alkoxy, C₁-C₆-haloalkoxy, C₁-C₈-alkylamino or di-(C₁-C₈-alkyl)amino;
A′ is hydrogen, C₁-C₈-alkyl or C₁-C₆-haloalkyl;
p is 0, 1 or 2;
m is 0, 1, 2, 3, 4 or 5;
X is cyano, C₁-C₄-alkyl, C₁-C₄-haloalkyl, C₁-C₄-alkoxy or C₁-C₂-haloalkoxy;
R¹,R² independently of one another are hydrogen, C₁-C₈-alkyl, C₁-C₈-haloalkyl, C₃-C₆-cycloalkyl, C₃-C₆-halocycloalkyl, C₂-C₈-alkenyl, C₄-C₁₀-alkadienyl, C₂-C₈-haloalkenyl, C₃-C₆-cycloalkenyl, C₂-C₈-alkynyl, C₂-C₈-haloalkynyl or C₃-C₆-cycloalkynyl, phenyl, naphthyl, or a five- to ten-membered saturated, partially unsaturated or aromatic heterocycle which contains one to four heteroatoms from the group consisting of O, N and S,
R¹ and R² together with the nitrogen atom to which they are attached may also form a five- or six-membered ring which may be interrupted by an atom from the group consisting of O, N and S and/or may carry one or more substituents from the group consisting of halogen, C₁-C₆-alkyl, C₁-C₆-haloalkyl and oxy-C₁-C₃-alkyleneoxy or in which a nitrogen atom and an adjacent carbon atom may be linked by a C₁-C₄-alkylene chain;
where R¹ and/or R² may be substituted by one to four identical or different groups R^{a}:
R^{a} is halogen, cyano, nitro, hydroxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₂-C₆-alkenyl, C₂-C₆-alkenyloxy, C₃-C₆-alkynyloxy, C₃-C₆-cycloalkyl, phenyl, naphthyl, a five- to ten-membered saturated, partially unsaturated or aromatic heterocycle which contains one to four heteroatoms from the group consisting of O, N and S,
where these aliphatic, alicyclic or aromatic groups for their part may be partially or fully halogenated or may carry one to three groups R^{b}:
R^{b} is halogen, cyano, nitro, hydroxyl, mercapto, amino, carboxyl, aminocarbonyl, aminothiocarbonyl, alkyl, haloalkyl, alkenyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl or alkynyl groups in these radicals contain 2 to 8 carbon atoms; and/or one to three of the following radicals:
cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated or substituted by alkyl or haloalkyl groups; and
R³ is cyano, hydroxyl, C₁-C₈-alkoxy, C₃-C₈-alkenyloxy, C₁-C₈-haloalkoxy, C₃-C₈-haloalkenyloxy, NR¹R² or S(O)ₙR³¹;
n is 0, 1 or 2;
R³¹ is hydrogen, hydroxyl, C₁-C₈-alkyl, C₂-C₈-alkenyl or —C(═O)-A.

2. A compound of the formula I.1, 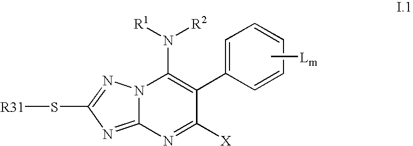
in which the variables and the index m are as defined for formula I as claimed in claim 1.

3. A compound of the formula I.2 or I.3, 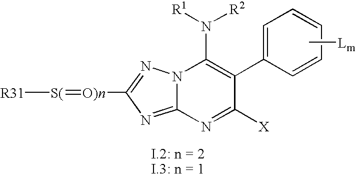
in which the variables and the index m are as defined for formula I as claimed in claim 1.

4. A compound of the formula IA, 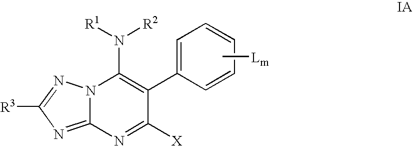
in which
R³ is cyano, hydroxyl, C₁-C₈-alkoxy, C₃-C₈-alkenyloxy, C₁-C₈-haloalkoxy, C₃-C₈-haloalkenyloxy or NR¹R²;
and R¹, R², X and Lₘ are as defined for formula I according to claim 1.

5. A compound of the formula I as claimed in any of claims 1 to 4, in which R¹ and R² are as defined below:
R is C₁-C₆-alkyl, C₁-C₈-haloalkyl, C₃-C₆-cycloalkyl, C₃-C₆-halocycloalkyl, C₂-C₈-alkenyl, C₂-C₈-haloalkenyl, C₂-C₈-alkynyl; and
R² is hydrogen or C₁-C₄-alkyl; or
R¹ and R² together with the nitrogen atom to which they are attached may also form a five- or six-membered saturated or unsaturated ring which may carry one or two substituents from the group consisting of halogen, C₁-C₆-alkyl and C₁-C₆-haloalkyl.

6. A compound of the formula I as claimed in claim 1, in which the phenyl group substituted by Lₘ is the group A 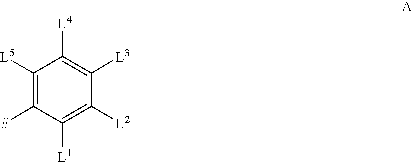
in which # is the point of attachment to the triazolopyrimidine skeleton and
L¹ is fluorine, chlorine, CH₃ or CF₃;
L²,L⁴ independently of one another are hydrogen or fluorine;
L³ is hydrogen, fluorine, chlorine, cyano, CH₃, SCH₃, OCH₃, SO₂CH₃ or COOCH₃ and
L⁵ is hydrogen, fluorine or CH₃.

7. Process for preparing the compounds of the formula I.1, as claimed in claim 2, in which X is cyano, C₁-C₄-alkoxy or C₁-C₂-haloalkoxy, by reacting compounds of the formula II 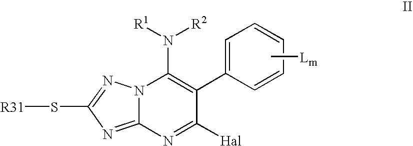
in which Hal is a halogen atom with compound M-X′ 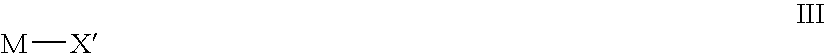
in which M in formula III is an ammonium, tetraalkylammonium or alkali metal or alkaline earth metal and X′ is cyano, C₁-C₄-alkoxy or C₁-C₂-haloalkoxy.

8. A process for preparing the compounds of the formula I.1 as claimed in claim 2 in which X is C₁-C₄-alkyl, by reacting compounds II as set forth in claim 7 and malonates of the formula IV, 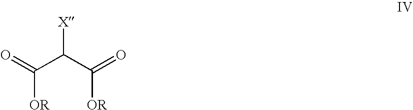
in which X″ is hydrogen or C₁-C₃-alkyl and R is C₁-C₄-alkyl to give compounds of the formula V 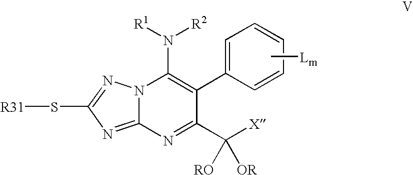
and hydrolysis of V and decarboxylation.

9. A process for preparing the compounds of the formula I.1 as claimed in claim 2 in which X is C₁-C₄-alkyl or C₁-C₄-haloalkyl, by reacting triazoles of the formula VI 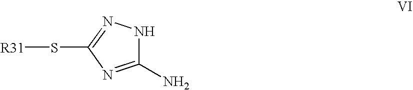
with dicarbonyl compounds of the formula VII, 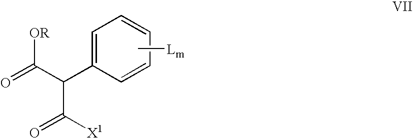
in which R and X¹ are or C₁-C₄-haloalkyl, to give hydroxytriazolopyrimidines of the formula VIII, 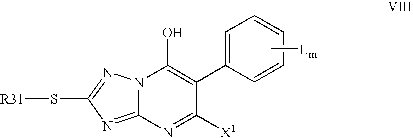
halogenation of VIII to give compounds of the formula IX 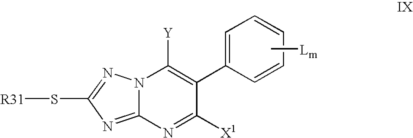
in which Y is halogen, in particular chlorine or bromine, and reaction of IX with amines of the formula X, 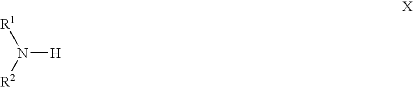
in which R¹ and R² are as defined in claim 1.

10. A process for preparing compounds of the formula I.1 as claimed in claim 2, in which X is C₁-C₄-alkyl, by reacting halogen compounds of the formula II as set forth in claim 7 with organometallic reagents of the formula XI
My(-X)y XI
in which M is B, Zn or Sn, X is C₁-C₄-alkyl and y corresponds to the valency of M.

11. A process for preparing compounds of the formula I.2 as claimed in claim 3 by oxidizing compounds of the formula I.1 as claimed in claim 2.

12. A process for preparing compounds of the formula IA as claimed in claim 4, by reacting compounds of the formula I.2 as claimed in claim 3 in which n=2 with compounds of the formula XII 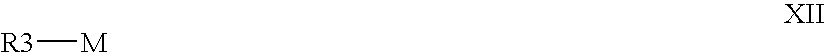
in which M is an ammonium, tetraalkylammonium, alkali metal or alkaline earth metal cation and R³ is as defined for formula IA, under basic conditions.

13. A composition suitable for controlling harmful fungi, which composition comprises a solid or liquid carrier and a compound of the formula I as claimed in claim 1.

14. A method for controlling phytopathogenic harmful fungi, which method comprises treating the fungi or the materials, plants, the soil or seeds to be protected against fungal attack with an effective amount of a compound of the formula I as claimed in claim 1.
